(19) United States
(12) Patent Application Publication
(10) Pub. No.: US 2014/0187937 A1
(43) Pub. Date: Jul. 3, 2014

(54) ULTRASOUND PROBE SWITCHOVER DEVICE AND A CORRESPONDING ULTRASOUND IMAGING SYSTEM

(71) Applicant: GE MEDICAL SYSTEMS GLOBAL TECHNOLOGY COMPANY, LLC, Waukesha, WI (US)

(72) Inventors: ZHENYU LIU, Wuxi (CN); Hangjun LI, WuXi (CN)

(73) Assignee: GE MEDICAL SYSTEMS GLOBAL TECHNOLOGY COMPANY, LLC, Waukesha, WI (US)

(21) Appl. No.: 14/137,266

(22) Filed: Dec. 20, 2013

(30) Foreign Application Priority Data
Dec. 28, 2012 (CN) 201210597854.3

(51) Int. Cl. A61B 8/00

(52) U.S. Cl. CPC A61B 8/56; A61B 8/44
USPC 600/437

(57) ABSTRACT

An ultrasound probe switchover device and a corresponding ultrasound imaging system, the device comprising connecting lines configured to directly connect signal pins of ultrasound probes and signal channels of a transmit-receive beamformer, wherein each connecting line first connects together a set of signal pins with the same number in the ultrasound probes, and then connects the set to a corresponding signal channel of the transmit-receive beamformer.

### TECHNICAL FIELD

Embodiments of the present invention relate to the technical field of medical equipment, and in particular to the technical field of ultrasound imaging system.

### BACKGROUND ART

A medical ultrasound imaging system generally provides two or more different probes for alternate use by a user. Therefore, as shown in FIG. 1, there is a need for a 1-in-N switching circuit 101 to select, among N probes, a probe 102 to be used to connect with an ultrasound beamformer 103.

A beamformer 103 of a portable or low-end ultrasound imaging system generally has 64 or even less channels, but a typical ultrasound probe 104 mostly has 128 transducer elements, which requires a multiplexer circuit (MUX) module 105 to achieve the mapping connection between the signal pins 108 of the probe and an ultrasound beamformer 103.

If the number of transducer elements 107 of an ultrasound probe 102 among a plurality of probes in an ultrasound imaging system is more than 128 (e.g., 192), it is required that another multiplexer circuit (MUX) module 106 is added in the probe to achieve the mapping connection between the transducer elements 107 of the probe and the signal pins 109 of the probe, since the number of the signal pins 109 is only 128.

Most multiplexer circuits in the prior art are implemented in high-voltage analog switchover chips. 1-in-N switching circuits are typically implemented by relays or high-voltage analog switchover chips. The implementation by use of relay would increase the volume and power consumption of the whole equipment, while the implementation by use of high-voltage analog switchover chip would increase the equipment cost and cause higher loss of ultrasound signal.

### SUMMARY OF THE INVENTION

Embodiments of the present invention provide an ultrasound probe switchover device and a corresponding ultrasound imaging system, in which the 1-in-N switching circuit can be removed from the switchover device and the number of multiplexer circuits can be reduced, in order to solve the problem of requiring too many multiplexer circuits and multipath selection switches in the ultrasound probe switchover device in the prior art.

According to an embodiment of the present invention, there is provided an ultrasound probe switchover device comprising, connecting lines for directly connecting signal pins of ultrasound probes and signal channels of a transmit-receive beamformer, wherein each connecting line first connects together a set of signal pins with the same number in the ultrasound probes, and then connects them to a corresponding signal channel of the transmit-receive beamformer.

In an embodiment there is provided an ultrasound probe switchover device, wherein the number of signal pins of each ultrasound probe is equal to the number of channels of the transmit-receive beamformer.

In an embodiment there is provided an ultrasound probe switchover device comprising further one or more multiplexer circuit module, each multiplexer circuit module being located in an ultrasound probe in which the number of ultrasound transducer elements is larger than the number of the channels of the transmit-receive beamformer, and being arranged between the ultrasound transducer elements and the signal pins of the ultrasound probe.

In an embodiment there is provided an ultrasound probe switchover device comprising further one or more multi-channel switching circuit module, each multi-channel switching circuit module being located in an ultrasound probe in which the number of ultrasound transducer elements is equal to or less than the number of the channels of the transmit-receive beamformer, and being arranged between the ultrasound transducer elements and the signal pins of the ultrasound probe.

In an embodiment there is provided an ultrasound probe switchover device comprising further a control module for controlling each of the multiplexer circuit modules or multi-channel switching circuit modules to be in normal operating mode or in off mode.

Embodiments of the present invention further provide an ultrasound imaging system comprising an ultrasound probe switchover device according to the present invention. That is, the ultrasound probe switchover device comprises: at least one connecting line configured to directly connect at least one signal pin of the ultrasound probes and at least one signal channel of a transmit-receive beamformer. Each of the at least one connecting line first connects a set of signal pins with the same number in the ultrasound probes, and then connects the set of signal pins to a corresponding signal channel of the transmit-receive beamformer.

Compared to the prior art, an ultrasound probe switchover device and a corresponding ultrasound imaging system provided by the present invention have the following technical benefits: removing the 1-in-N switch in the existing ultrasound probe switchover device and reducing the number of multiplexer circuits, facilitating improvement of qualities of transmitted and received signals and thus further improvement of final imaging quality; lower implementation cost; and lower power consumption, small packaging volume, being particularly applicable for compact and portable ultrasound imaging systems.

### BRIEF DESCRIPTION OF THE DRAWINGS

FIG. 1 is a schematic diagram of an existing ultrasound probe switchover device.

FIG. 2 is a schematic diagram of an ultrasound probe switchover device according to an embodiment of the present invention.

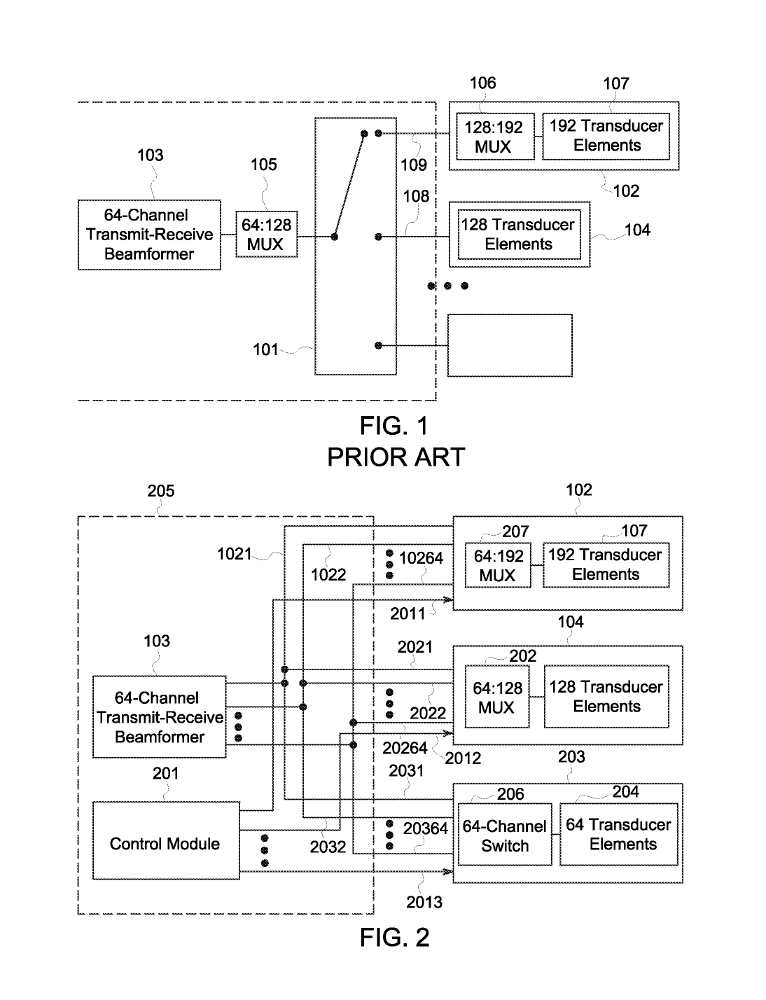

### DETAILED DESCRIPTION

Embodiments of the present invention will further be explained below in conjunction with accompanying drawings.

Some technical features well known to those skilled in the art are omitted for simplicity.

As shown in FIG. 2, in the embodiment, the transmit-receive beamformer 103 has 64 channels, the number of the transducer elements 107 in the ultrasound probe 102 is 192, the number of the transducer elements in the ultrasound probe 104 is 128, and the number of the transducer elements 204 in the ultrasound probe 203 is 64. There are 64 signal pins in each of the ultrasound probes. The transmit-receive beamformer 103 and the control module 201 are arranged in the ultrasound host 205.

For the probes 102 and 104 in which the number of the transducer elements is larger than the number of the channels of the transmit-receive beamformer 103, only multiplexer circuits (MUX) 207 and 202 are required to be respectively arranged in each probe. The multiplexer circuit (MUX) 207 is 64:192 multiplexer circuit; and the multiplexer circuit (MUX) 202 is 64:128 multiplexer circuit. Thus, the mapping connection between the transducer elements and the signal pins of the ultrasound probe may be achieved in one-shot, and since the number of signal pins of the ultrasound probe is equal to the number of the channels of the transmit-receive beamformer 103, the mapping between the transducer elements and the channels of the beamformer 103 is thus achieved in one-shot.

With respect to the probe 203 in which the number of transducer elements is equal to the number of the channels of the transmit-receive beamformer 103, the corresponding connection between the transducer elements and the signal pins of the ultrasound probe may be achieved simply by arranging a 64-channel switching circuit 206 in the probe.

With respect to the probe in which the number of transducer elements is less than the number of the channels of the transmit-receive beamformer 103, the corresponding connection between the transducer elements and the signal pins of the ultrasound probe may be achieved also by arranging within the probe a multi-channel switching circuit with the number of channels being not less than the number of the elements.

To remove the 1-in-N switch, signal pins of each ultrasound probe in the probe switchover device are connected in such a way that signal pins of each ultrasound probe are numbered from 1 to 64 respectively, and then the signal pins with the same number in each ultrasound probe are connected together through a connecting line to form a signal line which is further connected to the channel with the same number in the transmit-receive beamformer 103. For example, the signal pin 1021 numbered 1 in the ultrasound probe 102, the signal pin 2021 numbered 1 in the ultrasound probe 202 and the signal pin 2031 numbered 1 in the ultrasound probe 203 are first electrically connected together through a connecting line to form a wire which is then connected to the channel numbered 1 of the transmit-receive beamformer 103; the signal pin 1022 numbered 2 in the ultrasound probe 102, the signal pin 2022 numbered 2 in the ultrasound probe 202 and the signal pin 2032 numbered 2 in the ultrasound probe 203 are first electrically connected together through a connecting line to form a wire which is then connected to the channel numbered 2 of the transmit-receive beamformer 103; the signal pin 10264 numbered 64 in the ultrasound probe 102, the signal pin 20264 numbered 64 in the ultrasound probe 202 and the signal pin 20364 numbered 64 in the ultrasound probe 203 are first electrically connected together through a connecting line to form a wire which is then connected to the channel numbered 64 of the transmit-receive beamformer 103. 64 wires may be formed by sequentially connecting these lines, and respectively connected to 64 channels of the transmit-receive beamformer 103.

The control module 201 arranged within the ultrasound host 205 is used to respectively control the multiplexer circuits or multi-channel switching circuits in each probe, so that the multiplexer circuit(s) will be in normal operating state or off state and the switching circuit(s) will be in on or off state.

In actual use by a user, when user himself or a default logic of the ultrasound imaging system has selected a probe as the activated probe to be used, the control module 201 will send a control signal to multiplexer circuits or multi-channel switching circuits in other probes that are not used at that time so as to cause them to be in off state, that is, cause the connection between transducer elements of the probe and signal pins of the probe to be broken. Thus, only signal pins of the probe to be activated are connected to channels of the transmit-receive beamformer 103.

The ultrasound probe switchover device according to embodiments of the present invention improves qualities of transmitted and received signals, has low implementation cost, low power consumption, small packaging volume, and is particularly applicable for compact and portable ultrasound imaging systems.

It should be noted that the embodiments described above are merely illustrative rather than limiting, and those skilled in the art may design many alternative embodiments without departing from the scope of the appended claims. The verbs “include” or “comprise” used herein does not exclude the elements and steps other than the elements and steps set forth in the claims or the specification. The word “a” or “an” preceding an element does not exclude the presence of a plurality of such elements.

What is claimed is:

1. An ultrasound probe switchover device, comprising connecting lines for directly connecting signal pins of the ultrasound probes and signal channels of a transmit-receive beamformer, wherein each connecting line first connects together a set of signal pins with the same number in the ultrasound probes, and then connects said set to a corresponding signal channel of the transmit-receive beamformer.

2. The ultrasound probe switchover device according to claim 1, wherein number of the signal pins of each ultrasound probe is equal to number of the channels of the transmit-receive beamformer.

3. The ultrasound probe switchover device according to claim 2, further comprising one or more multiplexer circuit module, each multiplexer circuit module is located in an ultrasound probe in which number of ultrasound transducer elements is larger than the number of the channels of the transmit-receive beamformer, and arranged between the ultrasound transducer elements and the signal pins of the ultrasound probe.

4. The ultrasound probe switchover device according to claim 3, further comprising one or more multi-channel switching circuit module, each multi-channel switching circuit module is located in an ultrasound probe in which the number of ultrasound transducer elements is equal to or less than the number of the channels of the transmit-receive beamformer, and arranged between the ultrasound transducer elements and signal pins of the ultrasound probe.

5. The ultrasound probe switchover device according to claim 4, further comprising a control module for controlling each of the multiplexer circuit module or multi-channel switching circuit module to be in normal operating mode or in off mode.

6. An ultrasound imaging system comprising the ultrasound probe switchover device according claim 1.

7. An ultrasound probe switchover device for a plurality of ultrasound probes, wherein each ultrasound probe comprises a plurality of signal pins, wherein each signal pin has an identifier, the ultrasound probe switchover device comprising:
a transmit-receive beamformer comprising a plurality of signal channels; and
a plurality of connecting lines, each connecting line comprising a first connecting side and a second connecting side, wherein the first connecting side is configured to connect one or more signal pins having the same identifier, and the second connecting side is configured to connect to a corresponding signal channel of the plurality of signal channels.
